(19) United States
(12) Patent Application Publication
(10) Pub. No.: US 2020/0194110 A1
(43) Pub. Date: Jun. 18, 2020

(54) SYSTEM AND METHOD FOR PATIENT HISTORY-SENSITIVE STRUCTURED FINDING OBJECT RECOMMENDATION

(71) Applicant: KONINKLIJKE PHILIPS N.V., EINDHOVEN (NL)

(72) Inventors: Gabriel Ryan Mankovich, Boston, MA (US); Amir Mohammad Tahmasebi Maraghoosh, Arlington, MA (US); Lucas de Melo Oliveira, Wilmington, MA (US)

(21) Appl. No.: 16/349,313

(22) PCT Filed: Nov. 14, 2017
(86) PCT No.: PCT/EP2017/079096

§ 371 Date: May 13, 2019

Related U.S. Application Data
(60) Provisional application No. 62/425,094, filed on Nov. 22, 2016.

(51) Int. Cl. G16H 30/40; G16H 50/70

(52) U.S. Cl. CPC G16H 30/40; G16H 15/00; G16H 50/70

(57) ABSTRACT

System and method for generating structured finding object (“SFO”) description recommendations. The system and method are configured to extract a plurality of first findings for a patient, assemble the first findings into a timeline for the patient, extract a plurality of second findings for a population of patients, determine a set of timelines for the population of patients, and determine conditional probabilities of findings occurring for each of the second findings in the set of timelines. Further, the system and method are configured to display the SFO description recommendations for each of the first findings in the patient's timeline based, at least in part, on the conditional probabilities for the first finding.

Suggestion engines facilitate easy and intuitive human-computer interaction. Radiologists normally annotate objects of interests as part of radiology interpretation workflow to identify critical findings and followup recommendations. It has been recognized that annotation of radiological image findings will help increase the value of the radiology interpretation as it allows for structured persistence of the image semantics and re-use by downstream utilization. Conditional probability-enabled methods, such as those disclosed in U.S. Patent Application No. 62/364,937 may be used to efficiently propose structured multi-valued annotations based on prior annotations and contextual cues as well as appropriate user interaction devices. The contextual cues are obtained from the image interpretation environment and/or auxiliary engines, such as image processing engines. The algorithmics described in the above methods can be fine-tuned depending on the user interaction device employed.

### BACKGROUND

While these methods are a vast improvement over the traditional string-based methods (string-based methods fall short for annotating radiology findings since strings are insufficiently granular for re-use), these methods still have certain pitfalls. In particular, by modelling the relationships across a population of patients, the methods extract only fundamental relationships and can actually lose the context of the current patient being annotated. As adoption of structured reporting solutions is increasing, and methods are emerging for how to accomplish fast structured reporting in the workflow, a very significant challenge in the approach of the above methods is how to properly model the current context to ensure the system is suggesting the most appropriate structured finding descriptions to the radiologists at the right time for the current patient.

Since, as described above, individual patients do not correspond well to the ‘average patient’ that is modeled in a large database of structured finding objects, the present disclosure aims to solve this problem by disclosing a system and method for utilizing a large corpus of radiological reports in combination with the historical data of an individual patient in order to suggest more accurate structured finding object descriptions to the annotating radiologist. That is, the descriptions suggested by the present disclosure are to be more appropriate for the individual patient's current status at the time of the annotation.

### SUMMARY

In one exemplary embodiment, a system is provided for generating structured finding object (“SFO”) description recommendations. The system contains a processor and a display. The processor is configured to extract a plurality of first findings for a patient, assemble the first findings into a timeline for the patient, extract a plurality of second findings for a population of patients, determine a set of timelines for the population of patients, and determine conditional probabilities of findings occurring for each of the second findings in the set of timelines. The display displays the SFO description recommendations for each of the first findings in the patient's timeline based, at least in part, on the conditional probabilities for the first finding.

In another exemplary embodiment, a method is described for generating structured finding object (“SFO”) description recommendations. The method describes extracting a plurality of first findings for a patient, assembling the first findings into a timeline for the patient, extracting a plurality of second findings for a population of patients, determining a set of timelines for the population of patients, and determining conditional probabilities of findings occurring for each of the second findings in the set of timelines. The method further describes displaying the SFO description recommendations for each of the first findings in the patient's timeline based, at least in part, on the conditional probabilities for the first finding.

In another exemplary embodiment, a non-transitory computer readable storage medium with an executable program stored thereon is described. The program, when executed, instructs a processor to perform actions that include extracting a plurality of first findings for a patient, assembling the first findings into a timeline for the patient, extracting a plurality of second findings for a population of patients, determining a set of timelines for the population of patients, and determining conditional probabilities of findings occurring for each of the second findings in the set of timelines. The program further instructs a display to display the SFO description recommendations for each of the first findings in the patient's timeline based, at least in part, on the conditional probabilities for the first finding.

### BRIEF DESCRIPTION

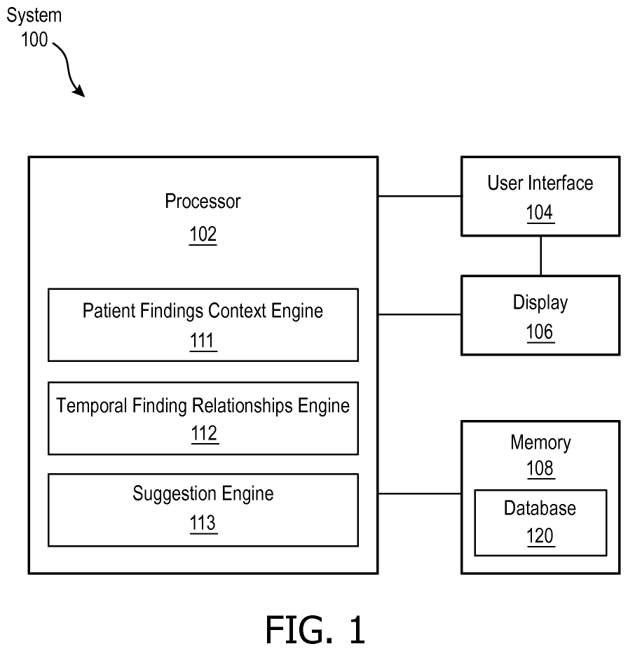

FIG. 1 shows a schematic drawing of a system according to an exemplary embodiment.

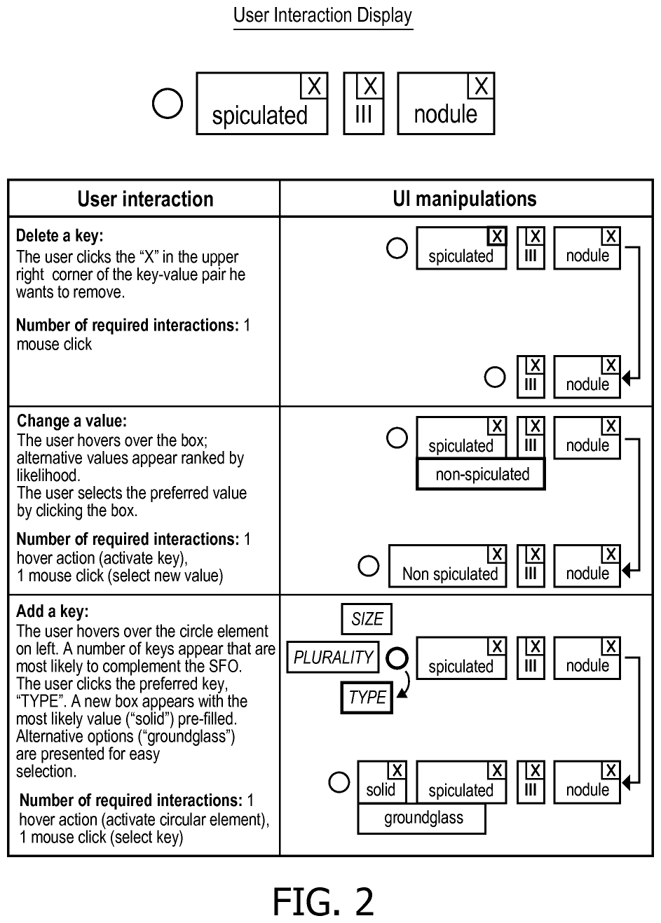

FIG. 2 shows a user interaction display, according to an exemplary embodiment.

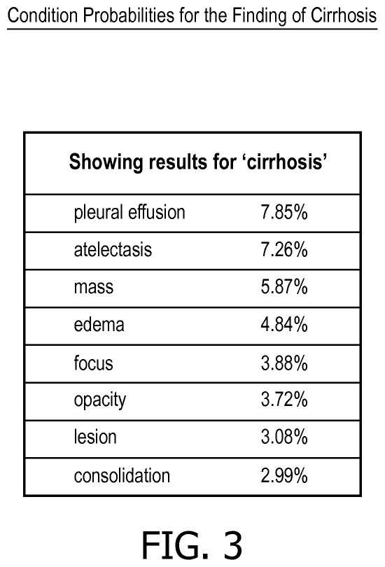

FIG. 3 shows conditional probabilities for the finding of cirrhosis, according to an exemplary embodiment.

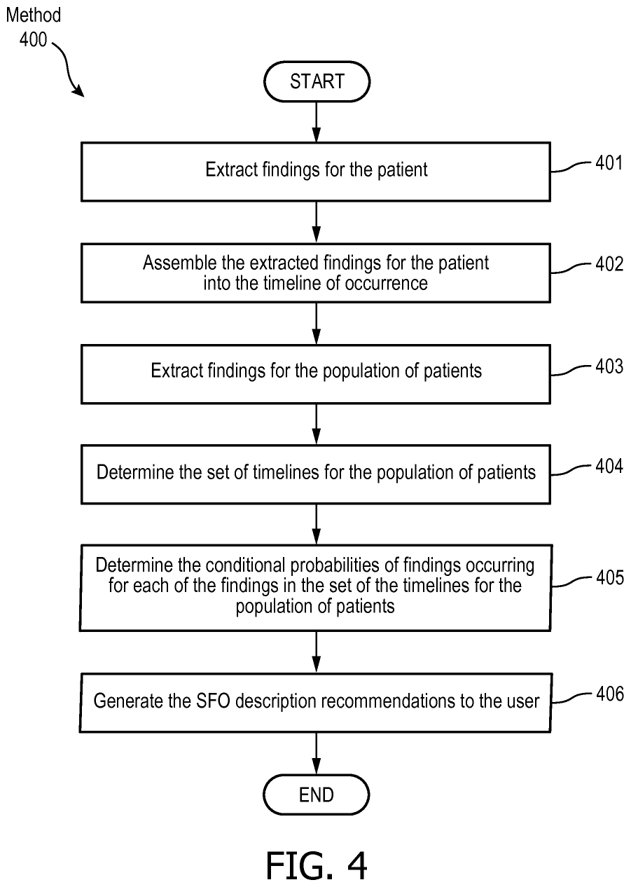

FIG. 4 shows a flow diagram of a method according to an exemplary embodiment.

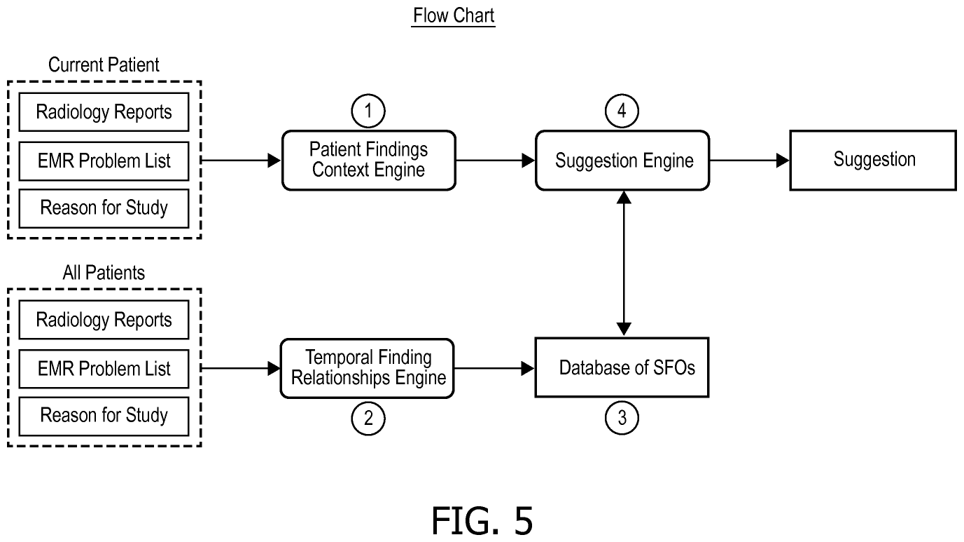

FIG. 5 shows a flow chart, according to an exemplary embodiment.

### DETAILED DESCRIPTION

The exemplary embodiments may be further understood with reference to the following description and the appended drawings, wherein like elements are referred to with the same reference numerals. The exemplary embodiments relate to a system and a method for providing a user with probability based contextual recommendations based on medical records from a plurality of patients and data from a patient whose medical record is being annotated. In particular, the exemplary embodiments suggest, to the user, structured finding object (SFO) descriptions, which will be discussed below, that better relate to the patient's current status at a time of the annotation.

The user may be a person tasked with annotating the medical record, such as a radiologist, a group of radiologists or any other person, medical professional, or group qualified to read the imaging scans from a Picture Archive and Communications System (PACS) or imaging system workstation. The medical records may include source documents, such as radiological reports, medical images, imaging scans, clinical reports, lab reports, etc.

The PACS is a workstation that aids radiologists in their duties and allows them to keep up with ever increasing workloads. In particular, the PACS employs an intuitive graphical user interface that provides access to the patient's radiological history, including diagnostic reports, exam notes, clinical history, and imaging scans. Further, the PACS has several features that simplify and speed up workflow. These features are critical in improving the radiologist's productivity.

The exemplary embodiments may be applied in any applications that involve annotating observation in imaging exams. For example, the exemplary embodiments may be utilized by systems such as Royal Philips Invivo DynaLync (a workflow solution for the integrated management of patient data associated with lung cancer screening) and IntelliSpace PACS Radiology.

Thus, it can be seen that the exemplary embodiments address a problem that is rooted in computer technology. Specifically, the issue of structured reporting did not exist prior to the ability to store the medical records in the memory of a computing device and store the data associated with the medical records in the computing device. Among other things, the exemplary embodiments solve the problem of accomplishing fast structured reporting by providing a system to suggest appropriate structured finding object descriptions based on a patient's medical history. Further, the exemplary embodiments significantly improve the productivity and efficiency of the medical professionals, in particular, the radiologists. As discussed above and will be further discussed below, the improvements to the productivity and efficiency of the radiologists is vital to both medical institutions and patients, alike, due to the constant rise in radiologist workloads.

The exemplary embodiments may be further understood with reference to the following description and the appended drawings, wherein like elements are referred to with the same reference numerals.

As shown in FIG. 1, a system 100, according to an exemplary embodiment of the present disclosure, is used to perform the exemplary functionalities that were described above. The system 100 comprises a processor 102, a user interface 104, a display 106, and a memory 108. Each of the components of the system 100 may include various hardware implementations. For example, the processor 102 may be a hardware component that comprises circuitry necessary to interpret and execute electrical signals fed into the system 100. Examples of processors 102 include central processing units (CPUs), control units, microprocessors, etc. The circuitry may be implemented as an integrated circuit, an application specific integrated circuit (ASIC), etc. The user interface 104 may be, for example, a keyboard, a mouse, a keypad, a touchscreen, etc. The display 106 may be a liquid crystal display (LCD) device, a light emitting diode (LED) display, an organic LED (OLED) display, a plasma display panel (PDP), etc. Those skilled in the art will understand that the functionalities of the user interface 104 and display 106 may be implemented in a single hardware component. For example, a touchscreen device may be used to implement both the display 106 and the user interface 104. The memory 108 may be any type of semiconductor memory, including volatile and non-volatile memory. Examples of non-volatile memory include flash memory, read only memory (ROM), programmable ROM (PROM), erasable PROM (EPROM) and electrically erasable PROM (EEPROM). Examples of volatile memory include dynamic random-access memory (DRAM), and fast CPU cache memory, which is typically static random-access memory (SRAM).

The memory 108 includes a database 120. The database 120 may store the medical records of the patient, the SFOs and temporal finding relationships. As discussed above, the medical records may include source documents, such as, radiological reports, imaging scans, medical images, clinical reports, lab reports, etc. Those of skill in the art will understand that the source documents may be written, oral or a combination of both. Further, the database 120 may store an electronic medical records (EMR) problems list for the patient and reason(s) for conducting a study. The temporal finding relationships may be provided by a temporal finding relationships engine 112. The temporal finding relationships engine 112 will be discussed below.

An SFO may comprise a set of key-value pairs {(k₁, v₁), . . . , (kₙ, vₙ)}, with a key kₙ representing a quantity which is observable from the medical record being annotated, and the value vₙ, representing a value of the image-observable quantity as may be observed from the medical record being annotated. In an exemplary embodiment, the SFO may be represented in a simplified form, namely as values {v₁, . . . , vₙ} of the key-value pairs, namely as “spiculated” and “nodule”. The corresponding key-value pairs may be {(spiculation, yes), (location, left lower lobe), (appearance, nodule)}.

An example of creating and modifying SFOs can be seen in FIG. 2. In particular, FIG. 2 shows how the user may create and modify an SFO for “spiculated left lower lobe nodule” through a variety of user interactions. For example, to delete a key value pair of “spiculated”, the user may click, e.g. using an onscreen pointer which is movable via a mouse, the “x” in the key value pair that the user seeks to remove, such as “spiculated”, as per FIG. 2. In another example, modification of a value of the key-value pair “spiculated” may be performed by the user hovering the onscreen pointer over a box representing the key-value pair “spiculated”. The display 106 may then show alternative values, which may be ranked by, for example, likelihood. Such likelihood may be determined by a probabilistic recommendation algorithm. As shown in FIG. 2, an alternative value of “non-spiculated” may be suggested.

In a final example, the addition of a key-value pair to the SFO may be performed by the user hovering the onscreen pointer over an addition symbol. The display 106 may display visual representation of a number of keys which are most likely to complement the SFO. As seen in FIG. 2, the user may select a preferred key, e.g., “TYPE”, and a new box may appear with a most likely value, “solid”, resulting in the SFO comprising a new key-value pair {type, solid}.

The SFO(s) may be stored on the database 120 in combination with contextual information. Such contextual information may be obtained from various sources, including but not limited to metadata of the medical records, image analysis information obtained from an image analysis of the medical report, an image viewer application enabling the user to view the medical image, and logging information of the system 100. A specific example of image analysis information is the anatomical label of selected voxels, or a probability distribution over anatomical locations assigned to each voxel by the image analysis. Another specific example is that the system 100 may ‘listen’ to an Application Programming Interface (API) of an image viewer application, e.g., as provided by the Picture Archiving and Communication System (PACS) viewing environment, to obtain contextual information in the form of detected user-initiated events.

Further examples of the SFOs, creating the SFOs, modifying the SFOs and storing the SFOs on the database 120 may be found in Patent Application No. 62/258,750 and U.S. Patent Application No. 62/364,937. Accordingly, U.S. Patent Application No. 62/258,750 and U.S. Patent Application No. 62/364,937 are hereby incorporated, in their entirety, by reference.

In an exemplary embodiment, the patient may be linked to a patient identifier. The patient identifier may be any type of an identification code, such as a Medical Record Number (MRN) or a Patient Identifier, used to identify the patient. The patient identifiers may also be stored in the database 120. The database 120 may be structured (e.g. in a structured format) in a manner that allows for patient specific queries. It should also be understood that the database 120 may represent a series of databases or other types of storage mechanisms that are distributed throughout system 100 or other interconnected systems.

The processor 102 may be implemented with engines, including, for example, a patient findings context engine 111, the temporal finding relationships engine 112, and a suggestion engine 113. Each of these engines will be described in greater detail below. Those skilled in the art will understand that the engines 111-113 may be implemented by the processor 102 as, for example, lines of code that are executed by the processor 102, as firmware executed by the processor 102, as a function of the processor 102 being an application specific integrated circuit (ASIC), etc.

The patient findings context engine 111 extracts findings that have occurred for the patient. The findings may be extracted from the database 120. For example, the findings may be extracted from the medical records of the patient. The findings may further be codified or the findings may be the SFOs. If the findings are codified, each of the findings may be associated with a specific code which correlates the finding to a predetermined code. In an exemplary embodiment, the extraction may be conducted by utilizing a concept extraction Natural Language Processing (“NPL”) pipeline on all radiological reports for the patient. Those skilled in the art would understand that the radiological reports may be of any specified type and that the extraction may be limited in scope. For example, the extraction may be limited by a time period (e.g., prior two years), specific dates or the like. In a further exemplary embodiment, the concept extraction NPL pipeline may be Medical Language Extraction and Encoding System (MedLEE) or National Center for Biomedical Ontology (NCBO) Annotator.

The patient findings context engine 111 further assembles the findings into a timeline of occurrence for the patient. In an exemplary embodiment, the assembling of the findings may be done by assigning a date of each of the radiological reports, or any other type of medical reports, to the findings found within the radiological reports. For example, extract coded (n) findings as a set (F) for each of the radiological reports (r) of the patient (p), where:

F_{p,r}=(f₁, f₂, . . . fₙ)

The sets may then be assembled into the timeline (T) for a total number of the radiological reports (m) for the patient (p) in a chronological order, where:

Tₚ=(F₁, F₂, . . . Fₘ)

The timeline may be stored in the database 120. In an exemplary embodiment, the patient findings context engine 111 may utilize the EMR problem list and/or the reasons for conducting the study along with or without the medical records to assemble the timeline.

The temporal finding relationships engine 112 runs the processes of the patient findings context engine 111 on a corpus of patients' medical records. In an exemplary embodiment, the corpus may consist of any number of the radiological reports of any number of the patients in the database 120, any number of the radiological reports of any number of the patients in a different database or a selected set of the patients based on specific criteria. For example, the criteria may limit the set to patients of a specific class (e.g., race, gender, age, nationality, etc.), a specific geographic region, a randomized or select quantity, a time period, etc.

The temporal finding relationships engine 112 may then determine a set of timelines from the corpus for each of the patients. Each of the timelines in the set of timelines may be determined by the processes discussed above regarding the patient findings context engine 111. From the set of timelines, the temporal finding relationships engine 112 may then determine conditional probabilities of a finding occurring for each of the findings in each of the set of timelines. That is, the conditional probabilities may be based on the findings occurring after a “prior finding”. An example of conditional probabilities may be seen in FIG. 3, which will be discussed below.

In an exemplary embodiment, the temporal finding relationships engine 112 may determine the probabilities for each or any of the prior findings by, first, assembling a set of all of the timelines (Tₐₗₗ) from the timelines of the patients (1), where:

Tₐₗₗ=(T₁, T₂, . . . Tₗ)

Second, the temporal finding relationships engine 112 may compute a probability of a finding (A) occurring for the prior finding (B) by counting a number of occurrences of the finding (A) in all of the timelines compared to the occurrences of the prior finding (B), where:

$$P ( {f}_{A} \vert {f}_{B} ) = \frac{{\sum }_{i = 1}^{l} {\sum }_{j = 1}^{m - 1} {\sum }_{k = j + 1}^{m} ( {f}_{B} , {f}_{A} ) ∈ {F}_{i , j} \times {F}_{i , k}}{{\sum }_{i = 1}^{l} {\sum }_{j = 1}^{m} ( {f}_{A} ∈ {F}_{i , j} )}$$

The probabilities may be stored in the database 120. As such, the database 120 may contain the probabilities of the findings occurring for each of the prior findings. In an exemplary embodiment, the temporal finding relationships engine 112 may utilize the EMR problem list and/or the reasons for conducting the study along with or without the corpus to compute the probabilities.

To illustrate, as seen in FIG. 3, for a prior finding of cirrhosis, the temporal finding relationships engine 112 may determine probabilities of findings occurring, such as a 7.85% chance of a pleural effusion [P(ƒ_{pleural effusion}±ƒ_{cirrhosis})] occurring for the patient having cirrhosis.

The suggestion engine 113 generates SFO description recommendations to the user based on the finding of the patient and the probabilities determined by the temporal finding relationships engine 112. For example, the suggestion engine 113 may recommend to the user, via the display 106, that the spiculated left lower lobe nodule, as discussed above, may be a pleural effusion, as seen in FIG. 3. In an exemplary embodiment, the suggestion engine 113 may display all or some of the results pertaining to the prior finding, with or without their percentages. The results may be limited by a predetermined cap, such as a number of probabilities to be shown, or by a predetermined threshold, such as a minimum percentage.

It should be noted that the suggestion engine 113 may include the functions disclosed in U.S. Patent Application No. 62/258,750, and the functions disclosed in U.S. Patent Application No. 62/364,937 may also be utilized in generating the SFO recommendations to the user.

FIG. 4 shows a method 400, according to an exemplary embodiment, for a patient history-sensitive SFO recommendation. FIG. 5 shows a flow chart to aid a visualization of method 400. In step 401, the patient findings context engine 111 may extract the findings for the patient. As discussed above, the finding may be extracted from the patient's medical records, such as from the radiological reports. In step 402, the patient findings context engine 111 may assemble the extracted findings into the timeline of occurrence for the patient. Steps 401 and 402 may correlate to bubble 1 of FIG. 5.

In step 403, the temporal finding relationship engine 112 may extract findings for a population of patients. The extraction may be conducted by running the processes of the patient findings context engine on the corpus of the patients' medical records, such as their radiological reports. As discussed above, the population may be limited by the criteria. In step 404, the temporal finding relationship engine 112 may determine a set of timelines for the population of patients.

In step 405, the temporal finding relationship engine 112 may determine the conditional probabilities of findings occurring for each of the findings (e.g., prior findings) in the set of the timelines for the population of patients. Steps 403, 404 and 405 may correlate to bubble 2 of FIG. 5.

In step 406, the suggestion engine 113 may generate SFO description recommendations to the user, such as the radiologist, based on the finding of the patient and the conditional probabilities for that finding as determined from the population of patients. As discussed above, the recommendations may be limited in scope by the predetermined cap or by the predetermined threshold. Step 406 may correlate to bubble 4 of FIG. 5.

It is noted that the claims may include reference signs/numerals in accordance with PCT Rule 6.2(b). However, the present claims should not be considered to be limited to the exemplary embodiments corresponding to the reference signs/numerals.

Those skilled in the art will understand that the above-described exemplary embodiments may be implemented in any number of ways, including, as a separate software modules, as a combination of hardware and software, etc. For example, the patient findings context engine 111, the temporal finding relationships engine 112, and the suggestion engine 113 may be programs containing lines of code that, when compiled, may be executed on a processor to perform the aforementioned functions.

1. A system for generating description recommendations relating to radiological imagery of a patient, comprising
a processor configured to:
extract a plurality of first findings for a the patient:
assemble the first findings into a timeline for the patient;
extract a plurality of second findings for a population of patients;
determine a set of timelines for the population of patients; and
determine conditional probabilities of findings occurring for each of the second findings in the set of timelines; and
a display that displays the description recommendations based, at least in part, on the first findings in the patient's timeline and the conditional probabilities for the second findings.

2. The system of claim 1, wherein the findings are extracted from medical records of the patient.

3. The system of claim 2, wherein the medical records comprise at least one of radiological reports, medical images, imaging scans, clinical reports or lab reports.

4. The system of claim 1, wherein the processor is further configured to: limit the population of patients by a criteria.

5. The system of claim 4, wherein the criteria comprises at least one of a race, a gender, an age group, a nationality, a geographic region, a time period, a randomized quantity, or a select quantity.

6. The system of claim 1, wherein the first and second findings are at least one of codified or structured.

7. The system of claim 1, wherein the description recommendations are numerically capped by a predetermined limit.

8. A method for generating description recommendations relating to radiological imagery of a patient, comprising:
extracting a plurality of first findings for patient;
assembling the first findings into a timeline for the patient,
extracting a plurality of second findings for a population of patients;
determining a set of timelines for the population of patients;
determining conditional probabilities of findings occurring for each of the second findings in the set of timelines; and
displaying the description recommendations based, at least in part, on the first findings in the patient's timeline and the conditional probabilities for the second findings.

9. The method of claim 8, wherein the findings are extracted from medical records of the patient.

10. The method of claim 9, wherein the medical records comprise at least one of radiological reports, medical images, imaging scans, clinical reports or lab reports.

11. The method of claim 8, further comprising:
limiting the population of patients by a criteria.

12. The method of claim 11, wherein the criteria comprises at least one of a race, a gender, an age group, a nationality, a geographic region, a time period, a randomized quantity, or a select quantity.

13. The method of claim 8, wherein the first and second findings are at least one of codified or structured.

14. The method of claim 8, wherein the description recommendations are numerically capped by a predetermined limit.

15. A non-transitory computer readable storage medium with an executable program stored thereon, wherein the program instructs a processor to perform actions for generating description recommendations relating to radiological imagery of a patient, that include:
extracting a plurality of first findings for the patient:
assembling the first findings into a timeline for the patient,
extracting a plurality of second findings for a population of patients;
determining a set of timelines for the population of patients;
determining conditional probabilities of findings occurring for each of the second findings in the set of timelines, and
displaying, on a display, the description recommendations based, at least in part, on the first findings in the patient's timeline and the conditional probabilities for the second findings.

16. A non-transitory computer readable storage medium of claim 15, wherein the findings are extracted from medical records of the patient.

17. A non-transitory computer readable storage medium of claim 16, wherein the medical records comprise at least one of radiological reports, medical images, imaging scans, clinical reports or lab reports.

18. A non-transitory computer readable storage medium of claim 15, wherein the processor is further configured to:
limiting the population of patients by a criteria.

19. A non-transitory computer readable storage medium of claim 18, wherein the criteria comprises at least one of a race, a gender, an age group, a nationality, a geographic region, a time period, a randomized quantity, or a select quantity.

20. A non-transitory computer readable storage medium of claim 15, wherein the first and second findings are at least one of codified or structured.
